(19) United States
(12) Patent Application Publication
(10) Pub. No.: US 2009/0098625 A1
(43) Pub. Date: Apr. 16, 2009

(54) SYSTEM AND METHOD FOR SEQUESTERING CARBON DIOXIDE AND BIOLOGICAL CONVERSION OF CARBON DIOXIDE THROUGH PHYTOGENIC PROCESSING INTO BIOGAS AND OXYGEN

(76) Inventor: Thomas S. Rothweiler, Phoenix, AZ (US)

Correspondence Address: WEISS & MOY PC, 4204 NORTH BROWN AVENUE, SCOTTSDALE, AZ, 85251, US

(21) Appl. No.: 11/873,312

(22) Filed: Oct. 16, 2007

(51) Int. Cl. C12P 5/02; C12P 1/04; C12P 3/00

(52) U.S. Cl. 435/167; 435/170; 435/168

(57) ABSTRACT

A method for converting carbon dioxide into various gases is disclosed. The method comprises the use of naturally-occurring methanogenic bacteria, like that contained in peat moss, to convert carbon dioxide into various gases through bioconversion utilizing phytogenic processes.

### BACKGROUND OF THE INVENTION

While the 21st Century will be a time of developing energy technologies based on renewable resources, lessening the dependence on fossil fuels, the most readily available and ubiquitous fossil fuel is, and will be continue to be coal. This abundant fuel can continue to power our industry and our cities through combustion, and can do it without adding greenhouse gasses and other harmful emissions into our atmosphere. Its emissions of heavy metals and carbon dioxide can be controlled and contained through new processes. This invention covers the sequestering of the carbon dioxide produced by combustion.

A secondary aspect of this invention will be the biological conversion of atmospheric carbon dioxide through the same sequestering and bioconversion processes that are to be used in for removing CO2 from coal-fired and other fossil fuel power plants.

Atmospheric carbon dioxide, by some estimations, has reached a point, brought on by the Industrial Revolution, of rapidly causing global climate change. Many suggestions have been made as to slowing the increase of atmospheric carbon dioxide, but virtually none have been made for the actual reduction of CO2 in the atmosphere. Sequestering CO2 in the ground, once it has been produced by the combustion process of fossil fuels, namely coal, has been promoted as a way of limiting the release of new CO2. If fossil fuels continue to be needed, it is certainly one measure that can be taken.

However, any method that is employed to reduce the output of CO2 does nothing to reduce the total CO2 already in the atmosphere, and it the primary driver of climate change on a global scale. Already we see it in the number and intensity of tropical storms and hurricanes. We see it in the melting of glaciers and sea ice in the polar regions. The earth is getting warmer, and we can only speculate what it ultimately means for mankind, perhaps in the not too distant future.

The process to sequester the carbon dioxide in plant material is a naturally occurring processes known as biomass conversion or simply bioconversion. Carbon dioxide is a source of carbon for plants, and is usually converted through photosynthesis. Peat moss is another plant material that has the ability to convert CO2 through a combination of biological catalysts and phytosynthesis. It does not utilize photosynthesis, since this material typically occurs in marshy areas in peak bogs where sunlight is minimal.

Peat bogs that formed during all ice ages act as natural carbon sinks. They convert atmospheric CO2 into plant material and oxygen through a bioconversion process using naturally occurring methanogenic bacteria and other anaerobic types. It would be possible to supplement these peat bogs, on a world-wide scale with bacteria and other microorganisms that are particularly suited for this bioconversion process.

The primary mechanism for the conversion of CO2 into other gasses, chiefly ethane (CH6), methane (CH4) and hydrogen (H2) is accomplished through bioconversion utilizing phytogenic processes. In this process, methanogenic bacteria, which thrive in an environment void of oxygen and sunlight, convert the CO2 into nutrients for the plant and into other gasses. There are many types of such bacteria, which are classed as anaerobic bacteria. vs. aerobic bacteria. Aerobic bacteria metabolize oxygen and produce CO2. Peat moss contains naturally occurring anaerobic bacteria, known as methanogenic bacteria. They utilize the CO2 as a nutrient source, and produce both methane and hydrogen as by-product gasses.

### FIELD OF THE INVENTION

CO2 is the chief greenhouse gas. Biological conversion can break the CO2 into elemental carbon and oxygen. The carbon becomes sequestered in the peat moss. The CO2 is converted into H2 and CH4 by methanogenic bacteria. In a coal-fired power plant, the actual output of the stack, minus the CO2 after coursing through a system holding the peat in place is H2 and CH4. Only some of the carbon is sequestered within the peat. Some is deposited as soot, while some is released into the atmosphere, depending on the factors of the process. These factors are amount and quality of the peat moss, power plant output, quality of the coal, other elements within the stack gas, particulates, length of contact, force of movement of the gas, quantity and efficiency of bioconversion bacteria. In free atmospheric CO2, these factors would be similar, without the concentrations form combustion.

The H2 and the CH4 (Dihydrogen and methane) has several uses. Initially, these gasses can be returned to the combustion process to augment the BTU efficiency of the furnaces. A second option exists to separate the carbon then create enriched hydrogen, which can then be a source of clean energy for use in automotive and other engines.

The length of travel for the carbon dioxide through the peat moss is a major factor, as will be the removal and replacement of peat moss that has absorbed and sequestered carbon to the point of saturation and super saturation. An internalized auger system can create an elongated pathway, maximizing the length of travel, while creating a method of removal and replenishment of the peat moss. The peat moss nearest the to the combustion chamber is removed first, while a continual fresh supply is added to the output end. The peat moss being removed has been exposed to the greatest amount of both heat and pressure, and might possibly have become a low-grade lignite coal in the process. Lignite is itself a low-grade coal.

Several alternative uses exist for the peat moss that has become saturated with carbon and any contaminants that have accumulated in the process. In nature, peat moss is considered to be a low grade coal, needing only additional eat and pressure to convert it to higher states of coal, with various contaminants being factors. Thus, this process could result in a manufactured coal, further contributing to the production of clean energy, since the contaminants from the initial combustible coal will be controlled. This peat can also be used as a soil amendment, returning valuable minerals and organic matter, not to mention carbon, to cropland.

Use sulfur hexafluoride gas (SP6) to absorb CO2 from the atmosphere. Create a corral above peat bogs that have been infused with methanogenic bacteria. This will contain the SF6. The sides of the corral must be high enough to prevent mixing of the SF6 with atmospheric air on windy days. Put the heavier than air gas into the coral, let it sink into the peat/spagnum moss, pulling the atmospheric CO2 into it.

### EMBODIMENTS

1. Fresh peat moss is harvested and placed into a mechanism that allows flu gasses to pass through.

2. The mechanism, which may be considered a collection tube, will have an internal mechanism that pushes the peat moss towards the incoming flue gasses.

3. The rate of forward motion of the peat moss is determined by the flue gas output, the concentrations of fly ash, heavy metals, the amount of CO2 and other factors existing within the combustion gasses and output.

4. In a free atmospheric carbon dioxide sequestering process, the mechanisms described in Embodiments 1, 2 and 3 would be similar, without combustion gasses.

5. The mechanism that moves the peat moss forward may be an auger.

6. As the peat moss moves toward the entrance of the stack, it becomes saturated, to where the most saturated peat is removed from the auger system.

7. The CO2 may stimulate peat moss growth, reducing the need for fresh peat moss to be added to the system.

8. If enough peat moss grows from the sequestering process, it may be a self-continuing cycle.

9. The off gassed ethane, methane and hydrogen is collected from the tube and fed directly into the furnace to add BTUs for increased power production.

10. The off-gassed ethane, methane and hydrogen is collected from the tube and processed for other than direct use in the furnace.

11. The off-gassed oxygen can be released into the atmosphere or cycled through a furnace.

12. The saturated peat moss is moved into continuous process anaerobic digesters for gasification of the peat moss.

13. The anaerobic digesters are infused with CH4 and H2, two essential nutrients for the existing methanogenic microorganisms.

14. The CO2 from the power plant provides the nutrients for the methanogenic bacteria already in the peat moss.

15. If there is still too much CO2 emitted from the stack, the peat moss can have supplementary methanogenic bacteria added.

16. The slurry of the depleted/activated peat moss can be de-watered and compressed into burnable blocks, being a low-grade lignite.

17. The depleted/activated peat moss can be used as a soil supplement.

18. If additional methanogenic bacteria are needed to supplement the sequestering and bioconversion systems, they can be produced in an anaerobic version of an OptiBac BioSystem.

19. The use of heavier-than-air gasses, mainly sulfur hexafluoride gas to draw atmospheric CO2 into existing, enriched peat bogs, to reduce total atmospheric CO2.

1. A method for converting carbon dioxide into various gases comprising using sphagnum peat moss containing naturally-occurring methanogenic bacteria to convert the carbon dioxide into various gasses, through bioconversion utilizing phytogenic processes.

2. The method of claim 1 wherein the carbon dioxide is converted to ethane (CH6), methane (CH4) and hydrogen (H2) through the bioconversion utilizing phytogenic processes.

3. The method of claim 1 further comprising injecting the sphagnum moss with methanogenic bacteria for bioconverting CO2 into other gasses.

4. The method of claim 1 further comprising directing CO2-rich effluents from coal-fired power plants, through the sphagnum moss, enriched with selected methanogenic bacteria.

5. The method of claim 1 further comprising containment of gasses produced by mathanogenic bioconversion of CO2 into flammable uses and for atmospheric release.

6. The method of claim 1 further comprising using SF6 (sulfur hexafluoride gas) as a carbon dioxide sink, drawing atmospheric CO2 into existing, enriched peat bogs, to reduce total atmospheric CO2.
